(19) United States
(12) Patent Application Publication
(10) Pub. No.: US 2016/0113860 A1
(43) Pub. Date: Apr. 28, 2016

(54) OIL-BASED THICKENING AGENT, OIL-BASED THICKENING COMPOSITION, AND COSMETIC PREPARATION

(71) Applicant: SHIN-ETSU CHEMICAL CO., LTD., Tokyo (JP)

(72) Inventor: Hiroko KIKUCHI, Tokyo (JP)

(73) Assignee: SHIN-ETSU CHEMICAL CO., LTD., Tokyo (JP)

(21) Appl. No.: 14/889,733

(22) PCT Filed: May 1, 2014
(86) PCT No.: PCT/JP2014/062044

§ 371 Date: Nov. 6, 2015

(30) Foreign Application Priority Data
May 9, 2013 (JP) 2013-099496

(51) Int. Cl. A61K 8/898; A61K 8/891; A61Q 1/10; A61Q 17/04; A61Q 5/06

(52) U.S. Cl. CPC A61K 8/898; A61Q 17/04; A61Q 5/06; A61Q 1/10; A61K 8/891; A61K 2800/48; A61K 2800/49; A61K 2800/592; A61K 2800/10

(57) ABSTRACT

Provided are: an oil-based thickening agent which is capable of providing an oil-based composition that has a high thickening effect on an oil component such as a silicone oil, while achieving excellent feeling of use; an oil-based thickening composition which contains this oil-based thickening agent and a liquid oil component; and a cosmetic preparation in which this oil-based thickening agent or oil-based thickening composition is blended. An oil-based thickening agent which is composed of a silicone-modified polysaccharide compound represented by formula (1) and a silicone emulsifying agent.

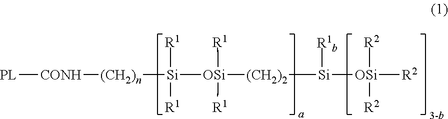

(In the formula, PL represents a glucose residue of pullulan; R¹ represents a monovalent hydrocarbon group having 1-10 carbon atoms; R² represents a monovalent hydrocarbon group having 1-10 carbon atoms or a siloxy group represented by —OSi(R³)₃; R³ represents a monovalent hydrocarbon group having 1-8 carbon atoms; n is an integer of 1-10; a is 0 or 1; and b is 0, 1 or 2. The average number of bonded silicone compounds (substitution degree of silicone compounds) per one constituent sugar unit of the polysaccharide compound is 0.5-2.5; and the average molecular weight of the silicone-modified polysaccharide compound is 50,000-10,000,000.)

### TECHNICAL FIELD

The present invention relates an oil-based thickening agent, especially an oil-based thickening agent which has a large thickening effect on oil-based ingredients and is capable of forming oil-based compositions that are pleasant to use; an oil-based thickening composition which contains the oil-based thickening agent and a liquid oil component; and a cosmetic preparation formulated with the oil-based thickening agent or the oil-based thickening composition.

In this invention, “oil-based thickening composition” refers to a composition which contains a specific oil-based thickening agent and a liquid oil component, which composition overall is of increased viscosity (thickened) compared with the viscosities of the respective compositions obtained by adding and blending, into the liquid oil component, the silicone-modified polysaccharide compound alone or the silicone emulsifying agent alone which are contained in the oil-based thickening agent.

### BACKGROUND ART

Because they spread easily and have a clean and light feel, silicone oils are used in a variety of cosmetic preparations and quasi-drugs, including skin care cosmetics, makeup cosmetics and hair cosmetics. At the same time, cosmetic preparations that use oil-based thickening agents include, for example, liquid foundations, sunscreen gels, moisturizing creams, hair gels and antiperspirant creams. However, there are substantially no oil-based thickening agents capable of thickening silicone oils and other oil-based ingredients which are satisfactory in terms of all relevant qualities such as thickening effect, pleasantness of use and stability.

Patent Document 1 (JP-A H05-311076) discloses that an polyether-modified silicone thickens silicone oil components in the presence of water.

However, when a polyether-modified silicone is used alone in the absence of water, the silicone oil thickening effect is very limited.

Patent Document 2 (JP-A H08-208989) discloses that silicone-modified pullulan, which is a silicone-modified polysaccharide, thickens silicone oil components, but this too has an inadequate thickening effect.

When polyether-modified silicones or silicone-modified polysaccharide compounds are included in large amounts within oil-based compositions or cosmetic preparations so as to increase the thickening effects, they are tacky or otherwise affect the pleasantness of use. Moreover, although both elicit thickening effects, those effects are inadequate.

### PRIOR ART DOCUMENTS

### Patent Documents

Patent Document 1: JP-A H05-311076

Patent Document 2: JP-A H08-208989

### SUMMARY OF THE INVENTION

### Problems to be Solved by the Invention

It is therefore an object of the present invention to provide an oil-based thickening agent which has a high thickening effect on silicone oils and other oil-based ingredients and is capable of forming oil-based compositions that are pleasant to use; an oil-based thickening composition which contains this oil-based thickening agent and a liquid oil component; and a cosmetic preparation formulated with the oil-based thickening agent or the oil-based thickening composition.

### Means for Solving the Problems

The inventors have conducted extensive investigations, as a result of which they have discovered that, by using an oil-based thickening agent which includes both a silicone emulsifying agent and a silicone-modified polysaccharide of formula (1), there can be obtained an oil-based thickening composition which has a high thickening effect on oil-based ingredients and which also is very pleasant to use.

Accordingly, the invention provides the following oil-based thickening agent, oil-based thickening composition and cosmetic preparation.

[1] An oil-based thickening agent comprising a silicone-modified polysaccharide compound of the general formula (1) below, and a silicone emulsifying agent:

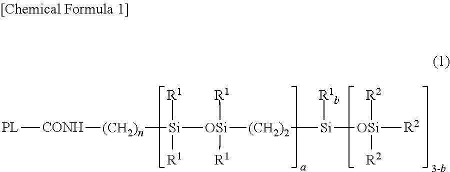

wherein PL is a glucose residue of pullulan; R¹ is identical or different and an unsubstituted or substituted monovalent hydrocarbon group of 1 to 10 carbon atoms; R² is identical or different and an unsubstituted or substituted monovalent hydrocarbon group of 1 to 10 carbon atoms or a siloxy group of the formula —OSi(R³)₃; R³ is identical or different and an unsubstituted or substituted monovalent hydrocarbon group of 1 to 8 carbon atoms; “n” is an integer from 1 to 10, “a” is 0 or 1, and “b” is 0, 1 or 2; and the average bonding number or degree of substitution of silicone compound per constituent sugar unit on the polysaccharide compound is from 0.5 to 2.5, and wherein the silicone-modified polysaccharide compound has an average molecular weight of from 50,000 to 10,000,000.
[2] The oil-based thickening agent according to [1], wherein the silicone-modified polysaccharide compound is a silicone-modified pullulan of the general formula (5) below:

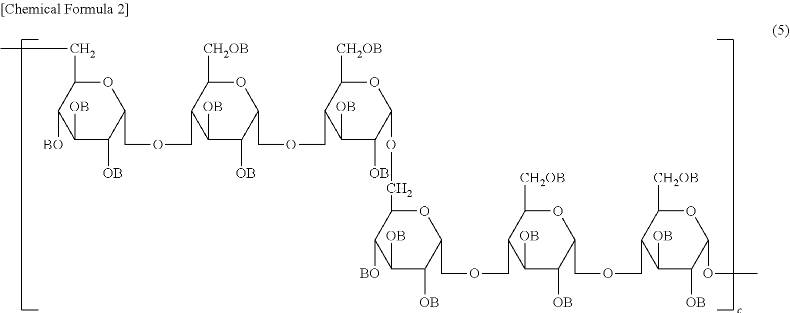

wherein B is a hydrogen atom or —CONH(CH₂)₃Si[OSi(CH₃)₃]₃, the degree of substitution is from 0.5 to 2.5, and “c” is a number from 100 to 20,000.
[3] The oil-based thickening agent according to [1] or [2], wherein the silicone emulsifying agent is one, two or more selected from among polyoxyalkylene-modified silicones and polyglycerol-modified silicones.
[4] An oil-based thickening composition characterized by comprising the oil-based thickening agent of any one of [1] to [3] and an oil-based ingredient.
[5] The oil-based thickening composition according to [4], wherein the oil-based ingredient is a liquid oil component.
[6] The oil-based thickening composition according to [5], wherein the oil-based liquid component is selected from among low-viscosity silicone oils having a kinetic viscosity of not more than 50 mm²/s, light isoparaffin and light liquid isoparaffin.
[7] A cosmetic preparation formulated with the oil-based thickening agent according to any one of [1] to [3] and/or the oil-based thickening composition according to any one of [4] to [6].

### Advantageous Effects of the Invention

According to this invention, by using an oil-based thickening agent which includes both a silicone emulsifying agent and a silicone-modified polysaccharide compound, there can be obtained an oil-based composition which exhibits a higher thickening effect on silicone oils and other oil-based ingredients than in cases where a silicone emulsifying agent or a silicone-modified polysaccharide compound is used alone. The resulting oil-based composition lacks tackiness and is pleasant to use, and thus lends itself well to use in cosmetic preparations.

In particular, by using one, two or more liquid oil components selected from among low-viscosity silicone oils having a kinetic viscosity of not more than 50 mm²/s, light isoparaffin and light liquid isoparaffin, an easy spreadability and a light, clean feel can be imparted to the oil-based thickening composition of the invention or the cosmetic preparation of the invention formulated with such an oil-based thickening agent or oil-based thickening composition.

### Embodiment for Carrying Out the Invention

The oil-based thickening agent of the invention is composed of a silicone-modified polysaccharide compound of the general formula (1) below having an average molecular weight of from 50,000 to 10,000,000, and a silicone emulsifying agent.

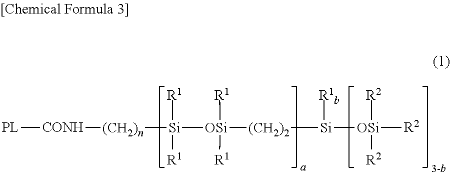

In formula (1), PL is a glucose residue of pullulan; R¹ is identical or different and an unsubstituted or substituted monovalent hydrocarbon group of 1 to 10 carbon atoms; R² is identical or different and an unsubstituted or substituted monovalent hydrocarbon group of 1 to 10 carbon atoms or a siloxy group of the formula —OSi(R³)₃; and R³ is identical or different and an unsubstituted or substituted monovalent hydrocarbon group of 1 to 8 carbon atoms. Also, “n” is an integer from 1 to 10, “a” is 0 or 1, and “b” is 0, 1 or 2. The average bonding number (degree of substitution) of silicone compound per constituent sugar unit on the polysaccharide compound is from 0.5 to 2.5.

### [Silicone-Modified Polysaccharide Compound]

The silicone-modified polysaccharide compound used in this invention is shown in the general formula (1) above.

In the general formula (1), PL is a glucose residue of pullulan. R¹ is identical or different and an unsubstituted or substituted monovalent hydrocarbon group of 1 to 10 carbon atoms, preferably 1 to 8 carbon atoms. R² is identical or different and an unsubstituted or substituted monovalent hydrocarbon group of 1 to 10 carbon atoms, preferably 1 to 8 carbon atoms, or a siloxy group of the formula —OSi(R³)₃, with R³ being identical or different and an unsubstituted or substituted monovalent hydrocarbon group of 1 to 8 carbon atoms, preferably 1 to 6 carbon atoms. Illustrative examples of these unsubstituted or substituted monovalent hydrocarbon groups R¹ to R³ include alkyl groups such as methyl, ethyl, propyl, isopropyl, butyl, isobutyl, tert-butyl, pentyl, hexyl, cyclohexyl, heptyl and octyl groups; alkenyl groups such as vinyl and allyl groups; aryl groups such as phenyl groups; and aralkyl groups such as benzyl groups; as well as any of these groups in which some or all of the hydrogen atoms have been substituted with, for example, halogen atoms such as fluorine, bromine or chlorine, or cyano groups. Examples include chloromethyl, chloropropyl, bromoethyl, trifluoropropyl and cyanoethyl groups. In terms of, for example, ease of synthesis and stability of the compound, it is desirable for these unsubstituted or substituted monovalent hydrocarbon groups represented by R¹ to R³ to be preferably unsubstituted or halogen-substituted monovalent hydrocarbon groups, especially unsubstituted monovalent hydrocarbon groups, and more preferably alkyl groups or aryl groups, especially methyl, ethyl or phenyl groups.

Also, “n” is an integer from 1 to 10, and preferably an integer from 1 to 6; “a” is 0 or 1, and preferably 0; and “b” is 0, 1 or 2, and preferably 0.

In the silicone-modified polysaccharide compound of the general formula (1) above, the average bonding number or degree of substitution of silicone compound per constituent sugar unit on the polysaccharide compound (pullulan) is from 0.5 to 2.5, and preferably from 1.0 to 2.0. When the average bonding number is too small, a sufficient oil solubility is not obtained; when it is too large, formation of the compound itself becomes difficult. The silicone compound of the general formula (2) below:

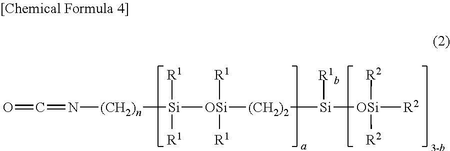

wherein R¹, R², “n”, “a” and “b” are the same as above.

In this invention, the “degree of substitution of a silicone-modified polysaccharide” refers to the average bonding number of silicone compound per constituent sugar unit on the polysaccharide compound. For example, the degree of substitution of the silicone-modified polysaccharide compound of the general formula (1) indicates the average number of the substituents shown in the general formula (4) below that are attached to the basic unit of pullulan shown in formula (3) below:

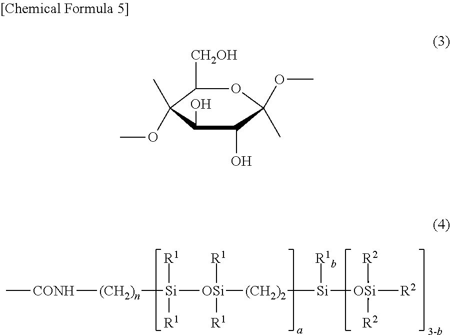

wherein R¹, R², “n”, “a” and “b” are the same as above.

Also, the silicone-modified polysaccharide compound in this invention has an average molecular weight of from 50,000 to 10,000,000, and preferably from 80,000 to 5,000,000. The average molecular weight can typically be determined as, for example, the polystyrene-equivalent number average molecular weight or weight average molecular weight in gel permeation chromatography analysis using toluene, tetrahydrofuran (THF) as the developing solvent.

In the silicone-modified polysaccharide compound of the invention, it is especially preferable for n=3, a=0, b=0 and R² to be methyl groups. Examples of such preferred silicone-modified polysaccharide compounds (i.e., cases in which n=3, a=0, b=0, and R² represents methyl groups) include the silicone-modified pullulan shown in the general formula (5) below.

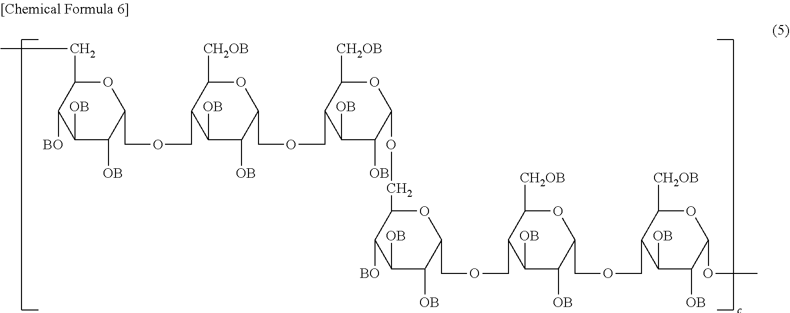

In the general formula (5), B is a hydrogen atom or a group of the formula —CONH(CH₂)₃Si[OSi(CH₃)₃]₃, the degree of substitution is from 0.5 to 2.5, and “c” is a number from 100 to 20,000.

The silicone-modified polysaccharide compound of formula (1) above that is used in this invention can be obtained by reacting an isocyanate-terminated silicone compound of the general formula (2) above with a polysaccharide compound (pullulan). A hitherto known method, such as that described in JP-A H08-134103, may be used to carry out such a reaction between a silicone compound and a polysaccharide compound.

The silicone-modified polysaccharide compound used in this invention can be obtained by a known method such as that mentioned above. For example, use may be made of the tri(trimethylsiloxy)silylpropylcarbamoyl pullulan (TSPL) defined in the Japanese Cosmetic Ingredient Labeling Name Dictionary (JCLD), commercial forms of which include TSPL-30-D5, which is TSPL dissolved in decamethylcyclopentasiloxane (available from Shin-Etsu Chemical Co., Ltd.), and TSPL-30-ID, which is TSPL dissolved in isododecane (available from Shin-Etsu Chemical Co., Ltd.).

### [Silicone Emulsifying Agent]

In the oil-based thickening agent of this invention, the thickening effect on oil-based ingredients is thought to appear as a result of the formation of a structure due to hydrogen bonding between hydrophilic groups on the silicone emulsifying agent and hydroxyl groups and urethane linkages (—OC(═O)NH—) on the silicone-modified polysaccharide compound. The silicone emulsifying agent used in this invention is not particularly limited, provided it is a silicone emulsifying agent capable of forming this structure. Use can be made of a silicone emulsifying agent that is known to the field of the invention.

Such silicone emulsifying agents are exemplified by polyoxyalkylene-modified silicones, polyglycerol-modified silicones and sugar alcohol-modified silicones. In cases where an especially high thickening effect is desired, the use of one, two or more selected from among polyoxyalkylene-modified silicones and polyglycerol-modified silicones is preferred.

Illustrative examples of polyoxyalkylene-modified silicones that may be used include the following defined in the Japanese Cosmetic Ingredient Labeling Name Dictionary (JCLD): PEG-3 Dimethicone, PEG-9 Methyl Ether Dimethicone, PEG-10 Dimethicone, PEG-9 Polydimethylsiloxyethyl Dimethicone, Lauryl PET-9 Polydimethylsiloxyethyl Dimethicone, PEG/PPG-18/18 Dimethicone, Cetyl PEG/PPG-10/1 Dimethicone, Dimethicone/(PEG-10/15) Crosspolymer, PEG-15/Lauryl Dimethicone Crosspolymer, PEG-10/Lauryl Dimethicone Crosspolymer, PEG-15/Lauryl Dimethicone Crosspolymer, and PEG-15/Lauryl Polydimethylsiloxyethyl Dimethicone Crosspolymer. These may be used as mixtures with an optional oil-based ingredient.

Commercial products include the following, available from Shin-Etsu Chemical Co., Ltd.: KF-6015, KF-6016, KF-6017, KF-6028, KF-6028P, KF-6038, KSG-210, KSG-240, KSG-310, KSG-320, KSG-330, KSG-340, KSG-320Z and KSG-350Z.

Illustrative examples of polyglycerol-modified silicones that may be used include the following defined in the Japanese Cosmetic Ingredient Labeling Name Dictionary (JCLD): Polyglyceryl-3 Polydimethylsiloxyethyl Dimethicone, Lauryl Polyglyceryl-3 Polydimethylsiloxyethyl Dimethicone, Bis-Butyl Dimethicone Polyglyceryl-3, Dimethicone/Polyglycerin-2 Crosspolymer, Lauryl Dimethicone/Polyglycerin-3 Crosspolymer and Polyglyceryl-3/Lauryl Polydimethylsiloxyethyl Dimethicone Crosspolymer. These may be used as mixtures with an optional oil-based ingredient.

Commercial products include the following, available from Shin-Etsu Chemical Co., Ltd.: KF-6104, KF-6105, KF-6109, KSG-710, KSG-810, KSG-820, KSG-830, KSG-840, KSG-820Z and KSG-850Z.

### [Oil-Based Thickening Agent]

The oil-base thickening agent of the invention is made up of the above silicone emulsifying agent and silicone-modified polysaccharide compound. Its thickening effect on oil-based ingredients is conspicuously high compared with cases in which the silicone emulsifying agent or the silicone-modified polysaccharide compound is used alone.

In the oil-based thickening agent of the invention, a thickening effect emerges when the silicone emulsifying agent and the silicone-modified polysaccharide compound are combined in any ratio. The ratio, expressed as silicone emulsifying agent: silicone-modified polysaccharide compound, is not particularly limited and may be varied over a broad range of from 0.5:99.5 to 99.5:0.5. The ratio of silicone emulsifying agent to silicone-modified polysaccharide compound is preferably from 1:4 to 4:1, and more preferably from 2:3 to 3:2.

### [Oil-Based Thickening Composition]

The oil-based thickening composition of the invention contains the oil-based thickening agent obtained above and an oil-based ingredient. The inventive oil-based thickening agent that uses a silicone emulsifying agent and a silicone-modified polysaccharide compound is able to thicken oil-based compositions or various oil-based ingredients formulated in cosmetic preparations. The various oil-based ingredients are not particularly limited, provided they are capable of dissolving the silicone emulsifying agent and the silicone-modified polysaccharide compound. Examples include oil-based ingredients commonly used in cosmetic preparations and external preparations for the skin, such as silicone oils, hydrocarbon oils, higher fatty acids, higher alcohols, ester oils, natural fatty oils, waxes and the like.

A liquid oil component may be suitably selected from among such liquid-based ingredients, particularly in cases where it is desired that the oil-based thickening composition which can be formulated with the oil-based thickening agent of the invention, or a cosmetic preparation formulated with the oil-based thickening agent or the oil-based thickening composition, spreads easily and has a clean, light feel. It is more preferable to use one, two or more selected from among low-viscosity silicone oils having a kinetic viscosity at 25° C. of not more than 50 mm²/s, and especially from 0.65 to 20 mm²/s, light isoparaffin and light liquid isoparaffin. The kinetic viscosity can be measured with a capillary kinematic viscometer.

Illustrative examples of such liquid oil components include the following defined in the Japanese Cosmetic Ingredient Labeling Name Dictionary (JCLD): Dimethicone, Cyclomethicone, Cyclopentasiloxane, Cyclohexasiloxane, Methyl Trimethicone, Diphenyl Dimethicone, Diphenylsiloxy Phenyl Trimethicone, Isododecane and Isohexadecane.

Commercial products include the KF-96 series, KF-995, TMF-1.5 and KF-56A, all available from Shin-Etsu Chemical Co., Ltd.; and Marukasol R, available from Maruzen Petrochemical Co., Ltd.

The oil-based thickening agent of the invention exhibits high thickening effects on these low-viscosity silicone oils, light isoparaffin and light liquid isoparaffin, enabling oil-based thickening compositions that are pleasant to use and have a good stability to be formed.

The oil-based thickening composition in this invention refers to a composition having a viscosity at least as large as (or larger than) when the silicone emulsifying agent and the silicone-modified polysaccharide compound are each dissolved alone in the liquid oil component. So long as the state of the composition has thickened, this includes compositions in the state of liquid substances ranging from low viscosity to high viscosity, and compositions in a gel state in which the liquid fluidity has been lost.

Preparation of the oil-based thickening composition of the invention is easy when the silicone emulsifying agent and the silicone-modified polysaccharide compound are separately dissolved in a low-viscosity silicone oil, light isoparaffin, light liquid paraffin or the like, and then mixed together. If necessary, heat may be applied during dissolution and mixture.

The amount of the oil-based thickening agent of the invention that is included in the oil-based composition may be selected as suitable for the intended purpose. Typically, from 0.1 to 80 wt % may be included, although the content is preferably from 3 to 50 wt %, and more preferably from 5 to 30 wt %. When the content is too low, sufficient effects are not exhibited. On the other hand, a content that is too high may make the composition tacky or otherwise less pleasant to use.

### [Cosmetic Preparation]

The inventive oil-based thickening agent composed of a silicone emulsifying agent and a silicone-modified polysaccharide compound is able, in small amounts, to thicken various oil-based ingredients, and can be advantageously used in cosmetic preparations without tackiness due to the thickening agent.

An oil-based thickening agent composed of a silicone emulsifying agent and a silicone-modified polysaccharide compound, or an oil-based composition obtained by adding various types of oil-based ingredients to an oil-based thickening agent, can be used in the cosmetic preparation of the invention.

The amount of the inventive oil-based thickening agent included in the cosmetic preparation may be set as appropriate for the intended purpose. Typically, based on the weight of the overall cosmetic preparation, from 0.1 wt % to 80 wt % may be included, with from 1 wt % to 50 wt % being preferred, and from 2 wt % to 30 wt % being especially preferred. When too little is included, a sufficient thickening effect may not be exhibited. On the other hand, when too much is included, this may impart undesirable effects such as tackiness to the cosmetic preparation.

The above-described oil-based composition may be used directly as the cosmetic preparation of the invention. However, in addition to the essential ingredients of the oil-based composition, where necessary, ingredients that are typically used in cosmetic preparations, such as moisturizers, ultraviolet absorbers, fragrances, antioxidants, preservatives, extender pigments, color pigments, alcohols and water, may be suitably included.

As to the form of the cosmetic preparation, the composition may be rendered into a nonaqueous system or into an oil-in-water or water-in-oil emulsified composition.

The cosmetic preparation is exemplified by skin care cosmetics such as lotions, emulsions, creams and serums; makeup cosmetics such as foundations, makeup bases, lipsticks, rouge, eye shadow, mascara and eyeliners; skin cleansers such as body soaps, face washes and makeup removers; hair cleansers such as shampoos; hair cosmetics such as rinses, hair treatments and hair growth preparations; and also hair dyes, sunscreen cosmetics and nail care cosmetics.

### EXAMPLES

Working Examples, Comparative Examples and example formulations are given below to more concretely illustrate the invention, although the invention is not limited by these Examples. Unless noted otherwise, the formulated amounts are indicated in % (wt %). The test methods used in the invention were as follows.

### (1) Viscosity

Samples having a viscosity of less than 1,000 mPa·s were measured with a Brookfield rotary viscometer (TV-10 viscometer (Toki Sangyo Co., Ltd.), rotor No. 2; speed, 60 rpm).

Samples having a viscosity of from 1,000 to 100,000 mPa·s were measured with a Brookfield rotary viscometer (TV10 viscometer (Toki Sangyo Co., Ltd.), rotor No. 4; speed, 6 rpm).

Samples having a viscosity of more than 100,000 mPa·s were measured with a Brookfield rotary viscometer (RVDV-III Ultra viscometer (Brookfield Engineering), spindle T-F; speed, 6 rpm).

### (2) Appearance

The sample was visually examined at room temperature (25° C.) and judged to be a solid when in a fluid state and a gel when in a non-fluid state.

### Working Examples and Comparative Examples

### Experiment 1

### Synergistic Effects from Concomitant Use

In oil-based compositions containing an oil-based thickening agent that uses a silicone emulsifying agent alone, a silicone-modified polysaccharide compound alone, or both, and containing also, as the liquid oil component, a silicone oil (decamethylcyclopentasiloxane), the concentration of oil-based thickening agent in the compositions was varied and the viscosities and appearances of the compositions were compared.

The materials used were as follows.

### Silicone Emulsifying Agent (1):

Polyglycerin-3 Polydimethylsiloxyethyl Dimethicone (KF-6104, from Shin-Etsu Chemical Co., Ltd.)

### Silicone-Modified Polysaccharide Compound (1):

Tri(trimethylsiloxy)silylpropylcarbamoyl pullulan (TSPL, from Shin-Etsu Chemical Co., Ltd.; degree of substitution in general formula (2), approx. 2.0; molecular weight, approx. 690,000).

The results are shown in Table 1.

TABLE 1

| | Comparative | | Comparative | Comparative | | Comparative |
|---|---|---|---|---|---|---|
| | Example 1 | Example 1 | Example 2 | Example 3 | Example 2 | Example 4 |
| Composition (%) | | | | | | |
| Silicone Emulsifying Agent (1) | 15 | 6 | 0 | 30 | 12 | 0 |
| Silicone-Modified | 0 | 9 | 15 | 0 | 18 | 30 |
| Polysaccharide Compound (1) | | | | | | |
| Decamethylcyclopentasiloxane | 85 | 85 | 85 | 70 | 70 | 70 |
| Oil-based thickening agent | 15 | | | 30 | | |
| concentration (%) in composition | | | | | | |
| Evaluation Results | | | | | | |
| Viscosity (mPa · s) | 14.5 | 10,300 | 203.4 | 48.2 | 262,000 | 11,000 |
| Appearance | liquid | liquid | liquid | liquid | gel | liquid |

As shown in Table 1, at an oil-based thickening agent content in the composition of 15%, with the use of Silicone Emulsifying Agent (1) alone (Comparative Example 1) or Silicone-Modified Polysaccharide Compound (1) alone (Comparative Example 2), the viscosity of the oil-based composition was very low, resulting in a liquid state having fluidity. By contrast, with an oil-based thickening agent according to the invention that uses two ingredients (Example 1), even though the oil-based thickening agent content in the composition was 15%, which is low, the viscosity improved relative to compositions in which only one of the oil-based thickening agent ingredients was used (Comparative Examples 1 and 2), thus demonstrating a synergistic thickening effect.

At an oil-based thickening agent content in the composition of 30%, with the use of Silicone Emulsifying Agent (1) alone (Comparative Example 3), as in the case where the content was 15%, the viscosity was very low. With the use of a Silicone-Modified Polysaccharide Compound (1) alone (Comparative Example 4), despite the increase in the oil-based thickening agent content from 15% to 30%, although some degree of thickening effect was apparent, the composition remained in the state of a liquid having fluidity. By contrast, with an oil-based thickening agent according to the invention that uses two ingredients (Example 2), a very hard gel lacking fluidity formed, thus demonstrating a marked thickening effect.

Therefore, by using Silicone Emulsifying Agent (1) and Silicone-Modified Polysaccharide Compound (1) together as the oil-based thickening agent, the viscosity of the oil-based composition markedly improved compared with cases in which either was used alone.

### Experiment 2

### Influence of Mixing Ratio

Aside from changing the mixing ratio of Silicone Emulsifying Agent (1) and Silicone-Modified Polysaccharide Compound (1), this investigation was carried out in the same way as in Experiment 1.

TABLE 2

| | Comparative | | | | | Comparative |
|---|---|---|---|---|---|---|
| | Example 1 | Example 3 | Example 4 | Example 1 | Example 5 | Example 2 |
| Composition (%) | | | | | | |
| Silicone Emulsifying Agent (1) | 15 | 12 | 9 | 6 | 3 | 0 |
| Silicone-Modified | 0 | 3 | 6 | 9 | 12 | 15 |
| Polysaccharide Compound (1) | | | | | | |
| Decamethylcyclopentasiloxane | 85 | 85 | 85 | 85 | 85 | 85 |
| Oil-based thickening agent | 15 | | | | | |
| concentration (%) in composition | | | | | | |
| Evaluation Results | | | | | | |
| Viscosity (mPa · s) | 14.5 | 1,900 | 9,400 | 10,300 | 3,000 | 203.4 |
| Appearance | liquid | liquid | liquid | liquid | liquid | liquid |

As shown in Table 2, oil-based thickening agents according to the invention which used two ingredients (Examples 1 and 3 to 5) had a synergistically improved viscosity over a broad range in mixing ratio.

### Experiment 3

### Type of Silicone Emulsifying Agent

This investigation was carried out in the same way as Experiment 1, but using silicone emulsifying agents of different structures.

The new materials used were as follows.

### Silicone Emulsifying Agent (2):

PEG-10 Dimethicone
(KF-6017, from Shin-Etsu Chemical Co., Ltd.)

### Silicone Emulsifying Agent (3):

A mixture of about 24% of Dimethicone/(PEG-10/15) Crosspolymer and about 76% of Methyl Polysiloxane (6 mm²/s) (KSG-210, from Shin-Etsu Chemical Co., Ltd.)

The results are shown in Table 3.

TABLE 3

| | Comparative | | Comparative | | Comparative | |
|---|---|---|---|---|---|---|
| | Example 5 | Example 6 | Example 6 | Example 7 | Example 7 | Example 8 |
| Composition (%) | | | | | | |
| Silicone Emulsifying Agent (2) | 15 | 6 | — | — | 30 | 12 |
| Silicone Emulsifying Agent (3) | — | — | 62.5¹⁾ | 25²⁾ | — | — |
| Silicone-Modified | 0 | 9 | 0 | 9 | 0 | 18 |
| Polysaccharide Compound (1) | | | | | | |
| Decamethylcyclopentasiloxane | 85 | 85 | 37.5 | 66 | 70 | 70 |
| Oil-based thickening agent | 15 | | | 30 | | |
| concentration (%) in composition | | | | | | |
| Evaluation Results | | | | | | |
| Viscosity (mPa · s) | 13.3 | 3,400 | 2,800 | 104,000 | 48.6 | 50,300 |
| Appearance | liquid | liquid | liquid | gel | liquid | liquid |

¹⁾Concentration of Dimethicone/(PEG-10/15) Crosspolymer in Silicone Emulsifying Agent (3) (KSG-210) was 15%
²⁾Concentration of Dimethicone/(PEG-10/15) Crosspolymer in Silicone Emulsifying Agent (3) (KSG-210) was 6%

As is apparent from Table 3, with regard to Silicone Emulsifying Agent (2), even when 15% (Comparative Example 5) or 30% (Comparative Example 7) of Silicone Emulsifying Agent (2) alone was included in the composition, the viscosity of the composition was very low. However, oil-based thickening agents according to the invention (Examples 6 and 8) that used Silicone Emulsifying Agent (2) and Silicone-Modified Polysaccharide Compound (1) together exhibited a high thickening effect. Moreover, as in Example 2, when 30% of an oil-based thickening agent according to the invention that is made up of the two ingredients was used (Example 8), an even higher thickening effect was exhibited.

Also, with regard to Silicone Emulsifying Agent (3), when Silicone Emulsifying Agent (3) was used alone (Comparative Example 6), this itself had an oil-based ingredient thickening effect. However, an oil-based thickening agent according to this invention (Example 7) that used Silicone Emulsifying Agent (3) and Silicone-Modified Polysaccharide Compound (1) together produced a hard gel, exhibiting a remarkable increase in viscosity.

Therefore, the effects of the invention are manifested between a plurality of types of silicone emulsifying agents and silicone-modified polysaccharide compounds.

### Experiment 4

### Type of Liquid Oil Component

Other than using, as liquid oil components other than decamethylcyclopentasiloxane: Isododecane, Diphenylsiloxy Phenyl Trimethicone (KF-56A, from Shin-Etsu Chemical Co., Ltd.; viscosity, 15 mm²/s) and Methyl Polysiloxane (KF-96A-6CS, from Shin-Etsu Chemical Co., Ltd.; viscosity, 6 mm²/s), this investigation was carried out in the same way as in Experiment 1.

The results are shown in Table 4.

TABLE 4

| | | | Example | Example | | Example | Example | Example |
|---|---|---|---|---|---|---|---|---|
| | Example 1 | Example 9 | 10 | 11 | Example 2 | 12 | 13 | 14 |
| Composition (%) | | | | | | | | |
| Silicone Emulsifying | 6 | 6 | 6 | 6 | 12 | 12 | 12 | 12 |
| Agent (1) | | | | | | | | |
| Silicone-Modified | 9 | 9 | 9 | 9 | 18 | 18 | 18 | 18 |
| Polysaccharide | | | | | | | | |
| Compound (1) | | | | | | | | |
| Decamethylcyclo- | 85 | — | 21 | 21 | 70 | — | 42 | 42 |
| pentasiloxane | | | | | | | | |
| Isododecane | — | 85 | — | — | — | 70 | — | — |
| KF-56A | — | — | 64 | — | — | — | 28 | — |
| KF-96A-6CS | — | — | — | 64 | — | — | — | 28 |
| Oil-based thickening | 15 | | | | 30 | | | |
| agent concentration | | | | | | | | |
| (%) in composition | | | | | | | | |
| Evaluation results | | | | | | | | |
| Viscosity (mPa · s) | 10,300 | 1,900 | 5,400 | 5,600 | 262,000 | 29,200 | 142,300 | 145,700 |
| Appearance | liquid | liquid | liquid | liquid | gel | liquid | gel | gel |

As shown in Table 4, even when Isododecane, KF-56A, or KF-96A-6CS was used instead of decamethylcyclopentasiloxane as the liquid oil component, oil-based thickening agents according to the invention that used Silicone Emulsifying Agent (1) and Silicone-Modified Polysaccharide Compound (1) together (Examples 9 to 14) all exhibited sufficient thickening effects.

Examples in which cosmetic preparations were produced using an oil-based thickening agent composed of a silicone emulsifying agent and a silicone-modified polysaccharide compound are shown below.

TABLE 5
Formulation Example 1: Emulsified liquid foundations

| | | 1-1 | 1-2 | 1-3 | 1-4 |
|---|---|---|---|---|---|
| (1) | Crosslinked polyether-modified silicone*¹ | — | 3.5 | 3.5 | 3.5 |
| (2) | Crosslinked dimethylpolysiloxane*² | 5.0 | 5.0 | 5.0 | 5.0 |
| (3) | Polyglycerol-modified silicone*³ | 4.0 | 2.0 | — | — |
| (4) | Branched polyether-modified silicone*⁴ | — | — | 2.0 | 10.0 |
| (5) | Dimethyldistearylammonium hectorite | 1.2 | 1.2 | 1.2 | 1.2 |
| (6) | Triethylhexanoin | 5.0 | 5.0 | 5.0 | 5.0 |
| (7) | Methyl polysiloxane (6 mm²/s) | 8.0 | 6.5 | 6.5 | 6.5 |
| (8) | Decamethylcyclopentasiloxane | 21.7 | 21.7 | 21.7 | 14.7 |
| (9) | 1,3-Butylene glycol | 5.0 | 5.0 | 5.0 | 5.0 |
| (10) | Sodium citrate | 0.2 | 0.2 | 0.2 | 0.2 |
| (11) | Sodium chloride | 0.5 | 0.5 | 0.5 | 0.5 |
| (12) | Purified water | 38.0 | 38.0 | 38.0 | 38.0 |
| (13) | Silicone-modified acrylic polymer*⁵ | 0.45 | 0.45 | 0.45 | 0.45 |
| (14) | Silicone-treated titanium oxide*⁶ | 8.50 | 8.50 | 8.50 | 8.50 |
| (15) | Silicone-treated red iron oxide*⁶ | 0.41 | 0.41 | 0.41 | 0.41 |
| (16) | Silicone-treated yellow iron oxide*⁶ | 0.97 | 0.97 | 0.97 | 0.97 |
| (17) | Silicone-treated black iron oxide*⁶ | 0.12 | 0.12 | 0.12 | 0.12 |
| (18) | Silicone-modified pullulan, 30% solution*⁷ | 1.0 | 1.0 | 1.0 | — |

Evaluation results

| | | | | |
|---|---|---|---|---|
| Viscosity (mPa · s) | 6,200 | 12,600 | 29,000 | 20,000 |
| Feel | Exc | Exc | Exc | NG |
| Stability | good | Exc | Exc | good |

*¹KSG-210, from Shin-Etsu Co., Ltd.
*²KSG-15, from Shin-Etsu Co., Ltd.
*³KF-6104, from Shin-Etsu Co., Ltd.
*⁴KF-6028P, from Shin-Etsu Co., Ltd.
*⁵KP-578, from Shin-Etsu Co., Ltd.
*⁶Treated with KF-9909, from Shin-Etsu Co., Ltd.
*⁷TSPL-30-D5, from Shin-Etsu Co., Ltd.

### Method of Evaluating Feel

Twenty expert panelists rated the feel in terms of the following five qualities.

1: Spreadability when applied

2: Tackiness

3: Evenness of finish

4: Sheerness of finish

5: Long-lasting (durability)

### Evaluation Criteria

Ratings were carried out based on the following criteria.
Exc: The foundation was satisfactory in terms of all five qualities.
Good: Only one of the five qualities was less than satisfactory.
NG: Two or more of the five qualities were less than satisfactory.

### Method of Evaluating Stability

High-temperature stability:
Evaluated after holding the foundation for 1 month in a 50° C. thermostatic chamber.

Normal-temperature stability:
Evaluated after holding the foundation for 1 month in a 50° C. thermostatic chamber.

Low-temperature stability:
Evaluated after holding the foundation for 1 month in a 50° C. thermostatic chamber.

### Evaluation Criteria

Ratings were carried out based on the following criteria.
Exc: The stability was excellent at all three temperature levels.
Good: The stability was good at all three temperature levels (even when the stability was excellent at up to two of the levels).
NG: The stability was poor (discoloration, change in odor, separation) at one or more of the three temperature levels.

Ingredients (1) to (8) were stirred and mixed to uniformity. In a separate step, ingredients (9) to (11) were uniformly dissolved in ingredient (12), following which the resulting solution was gently added to the mixture and emulsification was carried out. Ingredients (13) to (18), after being treated with rollers in a separate operation, were then added to and mixed with the emulsion. The resulting preparation was filled into a given container, giving an emulsified liquid foundation.

Formulations 1-1 to 1-4 were all prepared by the same method.

In Samples 1-1, 1-2 and 1-3 according to the invention in which a silicone emulsifying agent and a silicone-modified polysaccharide compound were used together, emulsified liquid foundations were obtained which, owing to the effects of the oil-based thickening agent, had a good stability, resisted running and smearing make-up due to human sebums (exhibited a high oil resistance), and were pleasant to use.

On the other hand, in Sample 1-4 formulated with only a silicone emulsifying agent, although the viscosity rose due to the increase in quantity of ingredient (4) and the stability was good, undesirable effects such as tackiness arose on account of the large amount of ingredient (4) added, and so this sample was judged to be unpleasant to use.

TABLE 6
Formulation Example 2: Sunscreen Emulsion
<Ingredients>

| | | |
|---|---|---|
| (1) | Crosslinked polyether-modified silicone*¹ | 3.0 |
| (2) | Crosslinked dimethylpolysiloxane*² | 2.0 |
| (3) | Branched polyether-modified silicone*³ | 1.0 |
| (4) | Silicone-modified pullulan, 30% solution*⁴ | 3.0 |
| (5) | Decamethylcyclopentasiloxane | 5.0 |
| (6) | Dimethylpolysiloxane (6 mm²/s) | 2.0 |
| (7) | Isotridecyl isononanoate | 4.0 |
| (8) | Titanium oxide dispersion*⁵ | 25.0 |
| (9) | Zinc oxide dispersion*⁶ | 35.0 |
| (10) | 1,3-Butylene glycol | 2.0 |
| (11) | Sodium citrate | 0.2 |
| (12) | Sodium chloride | 1.0 |
| (13) | Purified water | 16.8 |

*¹KSG-210, from Shin-Etsu Co., Ltd.
*²KSG-15, from Shin-Etsu Co., Ltd.
*³KF-6028P, from Shin-Etsu Co., Ltd.
*⁴TSPL-30-D5, from Shin-Etsu Co., Ltd.
*⁵SPD-T5, from Shin-Etsu Co., Ltd.
*⁶SPD-Z5, from Shin-Etsu Co., Ltd.

### <Production of Cosmetic Preparation>

A: Ingredients (1) to (7) were uniformly mixed together.
B: Ingredients (10) to (13) were mixed together.
C: B was added to A and emulsified, after which ingredients (8) and (9) were added, thereby giving a sunscreen emulsion.

The sunscreen emulsion thus obtained spread easily, was not tacky or oily, and had a good resistance to water and perspiration.

TABLE 7
Formulation Example 3: Sunscreen Lotion (shaking type)
<Ingredients>

| | | |
|---|---|---|
| (1) | Branched polyether-modified silicone*¹ | 2.0 |
| (2) | Silicone-modified pullulan, 30% solution*² | 3.0 |
| (3) | Dimethylpolysiloxane (6 mm²/s) | 7.0 |
| (4) | Decamethylcyclopentasiloxane | 5.8 |
| (5) | Ethylhexyl methoxycinnamate | 7.5 |
| (6) | Silicone hybrid powder*³ | 0.5 |
| (7) | Dimethyldistearylammonium hectorite | 0.2 |
| (8) | Zinc oxide dispersion*⁴ | 45.0 |
| (9) | 1,3-Butylene glycol | 3.0 |
| (10) | Alcohol | 5.0 |
| (11) | Sodium citrate | 0.2 |
| (12) | Sodium chloride | 0.5 |
| (13) | Purified water | 20.3 |

*¹KF-6028P, from Shin-Etsu Co., Ltd.
*²TSPL-30-D5, from Shin-Etsu Co., Ltd.
*³KSP-105, from Shin-Etsu Co., Ltd.
*⁴SPD-Z6, from Shin-Etsu Co., Ltd.

### <Production of Cosmetic Preparation>

A: Ingredients (1) to (7) were uniformly mixed together.
B: Ingredients (9) to (13) were mixed together.
C: B was added to A and emulsion was carried out, following which ingredient (8) was added, thereby giving a shaking type sunscreen lotion.

The sunscreen lotion thus obtained spread easily, was not tacky or oily, and was very long-lasting when applied.

TABLE 8
Formulation Example 4: Nonaqueous Mousse-type Foundation
<Ingredients>

| | | |
|---|---|---|
| (1) | Crosslinked polyether-modified silicone*¹ | 18.00 |
| (2) | Dimethylpolysiloxane (6 mm²/s) | 12.00 |
| (3) | Neopentyl glycol dioctanoate | 5.00 |
| (4) | Silyl-treated silicic anhydride*² | 0.75 |
| (5) | Silicone-treated red iron oxide*³ | 0.20 |
| (6) | Silicone-treated yellow iron oxide*³ | 1.00 |
| (7) | Silicone-treated black iron oxide*³ | 0.02 |
| (8) | Silicone-treated titanium oxide*³ | 5.00 |
| (9) | Silicone-treated talc*³ | 11.55 |
| (10) | Silicone-modified pullulan, 30% solution*⁴ | 3.00 |
| (11) | Trimethylsiloxysilicic acid, 50% solution*⁵ | 2.00 |
| (12) | Decamethylcyclopentasiloxane | 25.28 |
| (13) | Silicone hybrid powder*⁶ | 6.00 |
| (14) | Spherical polymethylsilsesquioxane*⁷ | 3.00 |
| (15) | Spherical poly(alkyl methacrylate) | 7.00 |
| (16) | Antioxidant | 0.20 |

*¹KSG-240, from Shin-Etsu Co., Ltd.
*²Aerosil R-972, a surface-hydrophobized fumed silica from Nippon Aerosil Co., Ltd.
*³Treated with KF-9909, from Shin-Etsu Co., Ltd.
*⁴TSPL-30-D5, from Shin-Etsu Co., Ltd.
*⁵KF-7312J, from Shin-Etsu Co., Ltd.
*⁶KSP-411, from Shin-Etsu Co., Ltd.
*⁷KMP-590, from Shin-Etsu Co., Ltd.

### <Production of Cosmetic Preparation>

Ingredients (1) to (8) were uniformly mixed together by treatment with rollers. Ingredients (9) to (16) were then added and mixing was carried out to uniformity, thereby giving a nonaqueous mousse-type foundation.

The foundation thus obtained had a firmly hardened appearance in the form of a mousse, spread easily and was very pleasant to use, without tackiness or oiliness. Moreover, it was confirmed to be very long-lasting when applied.

TABLE 9
Formulation Examples 5, 6 and 7: Mascara
<Ingredients>

| | | Formulation | Formulation | Formulation |
|---|---|---|---|---|
| | | Example 5 | Example 6 | Example 7 |
| (1) | Silicone-modified pullulan, 30% solution*¹ | 10.0 | 5.0 | 5.0 |
| (2) | Trimethylsiloxysilicic acid solution*² | — | 15.0 | — |
| (3) | Acrylic silicone resin solution*³ | — | — | 15.0 |
| (4) | Dextrin palmitate/ethylhexanoate*⁴ | 3.0 | 3.0 | 3.0 |
| (5) | Ceresin | 2.5 | 2.5 | 2.5 |
| (6) | Behenyl-modified acrylic silicone resin*⁵ | 2.0 | 2.0 | 2.0 |
| (7) | Beeswax | 4.5 | 4.5 | 4.5 |
| (8) | Hydrogenated lecithin | 0.4 | 0.4 | 0.4 |
| (9) | Triethylhexanoin | 2.5 | 2.5 | 2.5 |
| (10) | Branched polyether-modified silicone*⁶ | 1.0 | 1.0 | 1.0 |
| (11) | Dimethyldistearylammonium hectorite | 4.0 | 4.0 | 4.0 |
| (12) | Propylene carbonate | 1.3 | 1.3 | 1.3 |
| (13) | Isododecane | 42.6 | 32.6 | 32.6 |
| (14) | Silicone-treated pigment*⁷ | 5.0 | 5.0 | 5.0 |
| (15) | Silicone-treated talc*⁷ | 4.5 | 4.5 | 4.5 |
| (16) | Silylated silicic anhydride*⁸ | 2.7 | 2.7 | 2.7 |
| (17) | 1,3-Butylene glycol | 1.0 | 1.0 | 1.0 |
| (18) | Preservative | as suitable | as suitable | as suitable |
| (19) | Purified water | balance | balance | balance |

*¹TSPL-30-ID, from Shin-Etsu Co., Ltd.
*²X-21-5595, from Shin-Etsu Co., Ltd.
*³KP-550, from Shin-Etsu Co., Ltd.
*⁴Rheopearl TT, from Chiba Flour Milling Co., Ltd.
*⁵KP-562P, from Shin-Etsu Co., Ltd.
*⁶KF-6028, from Shin-Etsu Co., Ltd.
*⁷Treated with KF-9909, from Shin-Etsu Co., Ltd.
*⁸Aerosil R-972, a surface-hydrophobized fumed silica from Nippon Aerosil Co., Ltd.

### <Production of Cosmetic Preparation>

A: Ingredients (10) to (13) were uniformly mixed together.
B: Ingredients (14) to (16) were added to A, followed by uniform mixture by treatment with rollers.
C: Ingredients (1) to (9) were added to B, followed by warming and uniform mixture.
D: Ingredients (17) to (19), which were uniformly mixed in a separate operation, were warmed then added to C, followed by stirring to effect mixture, thereby giving a mascara.

Each of the mascaras thus obtained spread easily, readily adhered to the eyelashes, and was pleasant to use with no tackiness. In addition, secondary transfer did not readily occur (demonstrating a high rubbing resistance), and the mascara was confirmed to be very long-lasting.

Similar effects were confirmed to be achievable even when used together with silicone film formers commonly used in mascaras.

1. An oil-based thickening agent comprising a silicone-modified polysaccharide compound of the general formula (1) below, and a silicone emulsifying agent: 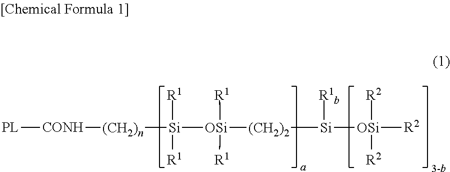
wherein PL is a glucose residue of pullulan; R¹ is identical or different and an unsubstituted or substituted monovalent hydrocarbon group of 1 to 10 carbon atoms; R² is identical or different and an unsubstituted or substituted monovalent hydrocarbon group of 1 to 10 carbon atoms or a siloxy group of the formula —OSi(R³)₃; R³ is identical or different and an unsubstituted or substituted monovalent hydrocarbon group of 1 to 8 carbon atoms; “n” is an integer from 1 to 10, “a” is 0 or 1, and “b” is 0, 1 or 2; and the average bonding number or degree of substitution of silicone compound per constituent sugar unit on the polysaccharide compound is from 0.5 to 2.5, and
wherein the silicone-modified polysaccharide compound has an average molecular weight of from 50,000 to 10,000,000.

2. The oil-based thickening agent according to claim 1, wherein the silicone-modified polysaccharide compound is a silicone-modified pullulan of the general formula (5) below: 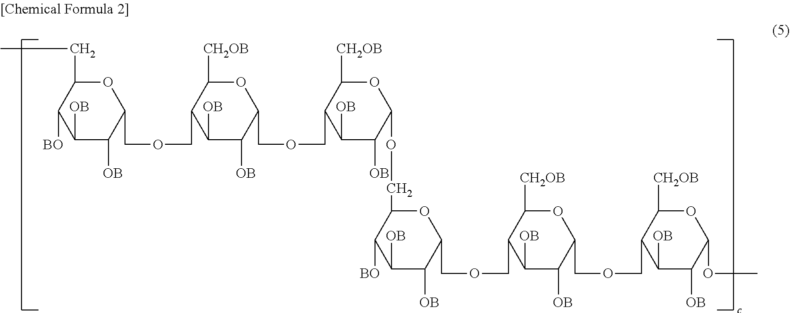
wherein B is a hydrogen atom or —CONH(CH₂)₃Si[OSi(CH₃)₃]₃, the degree of substitution is from 0.5 to 2.5, and “c” is a number from 100 to 20,000.

3. The oil-based thickening agent according to claim 1 or 2, wherein the silicone emulsifying agent is one, two or more selected from among polyoxyalkylene-modified silicones and polyglycerol-modified silicones.

4. An oil-based thickening composition characterized by comprising the oil-based thickening agent of claim 1 and an oil-based ingredient.

5. The oil-based thickening composition according to claim 4, wherein the oil-based ingredient is a liquid oil component.

6. The oil-based thickening composition according to claim 5, wherein the oil-based liquid component is selected from among low-viscosity silicone oils having a kinetic viscosity of not more than 50 mm²/s, light isoparaffin and light liquid isoparaffin.

7. A cosmetic preparation formulated with at least one of the oil-based thickening agent according to claim 1 or the oil-based thickening composition according to claim 4.
